(19) United States
(12) Patent Application Publication
(10) Pub. No.: US 2010/0191050 A1
(43) Pub. Date: Jul. 29, 2010

(54) VARIABLE LENGTH ACCESSORY FOR GUIDING A FLEXIBLE ENDOSCOPIC TOOL

(75) Inventor: Andrew M. Zwolinski, Cincinnati, OH (US)

Correspondence Address: K&L Gates LLP, 210 SIXTH AVENUE, PITTSBURGH, PA, 15222-2613, US

(73) Assignee: Ethicon Endo-Surgery, Inc., Cincinnati, OH (US)

(21) Appl. No.: 12/359,053

(22) Filed: Jan. 23, 2009

(51) Int. Cl. A61B 1/018; A61B 1/01

(52) U.S. Cl. 600/104; 600/114

(57) ABSTRACT

The present invention includes, in various embodiments, a surgical accessory for guiding a flexible endoscopic tool into an endoscope. In at least one embodiment, the surgical accessory includes a variable length assembly having a first component and a second component, a first connector, and a second connector. In such embodiments, the first component can be telescopically engaged with the second component, wherein the first and second components are adapted to insertably receive a flexible portion of a flexible endoscopic tool. In such embodiments, the first connector is configured to releasably connect the first component to the flexible endoscopic tool and the second connector is configured to releasably connect the second component to an endoscope.

### BACKGROUND

The present disclosure generally relates to surgical instruments and, more particularly, to an endoscope and endoscopic instruments that are positionable within a working channel of the endoscope.

Access to the abdominal cavity may, from time to time, be required for diagnostic and therapeutic endeavors for a variety of medical and surgical diseases. Historically, abdominal access has required a formal laparotomy to provide adequate exposure. Such procedures, which require incisions to be made in the abdomen, are not particularly well-suited for patients that may have extensive abdominal scarring from previous procedures, those persons who are morbidly obese, those individuals with abdominal wall infection, and those patients with diminished abdominal wall integrity, such as patients with burns and skin grafting. Other patients simply do not want to have a scar if it can be avoided.

Minimally invasive procedures are desirable because such procedures can reduce pain and provide relatively quick recovery times as compared with conventional open medical procedures. Many minimally invasive procedures are performed with an endoscope (including, without limitation, laparoscopes). Such procedures permit a physician to position, manipulate, and view medical instruments and accessories inside the patient through a small access opening in the patient's body. Laparoscopy is a term used to describe such an “endosurgical” approach using an endoscope (often a rigid laparoscope). In this type of procedure, accessory devices are often inserted into a patient through trocars placed through the body wall. Trocars must typically pass through several layers of overlapping tissue/muscle before reaching the abdominal cavity.

Still less invasive treatments include those that are performed through insertion of an endoscope through a natural body orifice to a treatment region. Examples of this approach include, but are not limited to, cholecystectomy, appendectomy, cystoscopy, hysteroscopy, esophagogastroduodenoscopy, and colonoscopy. Many of these procedures employ the use of a flexible endoscope during the procedure. Flexible endoscopes often have a flexible, steerable articulating section near the distal end that can be controlled by the user by utilizing controls at the proximal end. Minimally invasive therapeutic procedures to treat diseased tissue by introducing medical instruments to a tissue treatment region through a natural opening of the patient are known as Natural Orifice Translumenal Endoscopic Surgery (NOTES)TM.

Some flexible endoscopes are relatively small (about 1 mm to 3 mm in diameter), and may have no integral accessory channel (also called biopsy channels or working channels). Other flexible endoscopes, including gastroscopes and colonoscopes, have integral working channels having a diameter of about 2.0 mm to 3.5 mm for the purpose of introducing and removing medical devices and other accessory devices to perform diagnosis or therapy within the patient. As a result, the accessory devices used by a physician can be limited in size by the diameter of the accessory channel of the scope used. Additionally, the physician may be limited to a single accessory device when using the standard endoscope having one working channel.

Certain specialized endoscopes are available, such as large working channel endoscopes having a working channel of about 5 mm in diameter, which can be used to pass relatively large accessories, or to provide capability to suction large blood clots. Other specialized endoscopes include those having two or more working channels.

The above mentioned minimally invasive surgical procedures have changed some of the major open surgical procedures such as gall bladder removal, or a cholecystectomy, to simple outpatient surgery. Consequently, the patient's recovery time has changed from weeks to days. These types of surgeries are often used for repairing defects or for the removal of diseased tissue or organs from areas of the body such as the abdominal cavity.

The foregoing discussion is intended only to illustrate the present field of the invention and should not be taken as a disavowal of claim scope.

### SUMMARY

The present invention includes, in various embodiments, a surgical accessory for guiding a flexible endoscopic tool. In at least one embodiment, the surgical accessory includes a variable length assembly having a first component and a second component, a first connector, and a second connector. In these embodiments, the first component can be telescopically engaged with the second component, wherein the first and second components can be adapted to insertably receive a flexible portion of an endoscopic tool. Further, in these embodiments, the first connector is configured to releasably connect the first component to the flexible endoscopic tool and the second connector is configured to releasably connect the second component to an endoscope.

In at least one embodiment, a surgical accessory for guiding a flexible endoscopic tool is provided that includes a variable length assembly having a first end and a second end, a first connector, and a second connector. In these embodiments, the first end of the variable length assembly is movable relative to the second end, and the variable length assembly is configured to receive a flexible portion of the flexible endoscopic tool. Further, in these embodiments, the first connector is configured to releasably connect the first end of the variable length assembly to a flexible endoscopic tool and the second connector is configured to releasably connect the second end of the variable length assembly to an endoscope.

In at least one embodiment, a surgical instrument is provided that includes a variable length assembly for guiding a surgical tool and, in addition, a surgical tool for insertion into a patient's body. In these embodiments, the variable length assembly includes a first end that is movable relative to a second end. The first end is coupled to the surgical tool and the variable length assembly is configured such that movement of the surgical tool causes movement of the first end relative to the second end of the variable length assembly.

In at least one embodiment, a surgical instrument for insertion into a person's body is provided that includes a variable length assembly for guiding a surgical tool, in addition, and an endoscope having an insertable portion and a non-insertable portion. In these embodiments, the variable length assembly includes a first end that is movable relative to a second end wherein the first end is attached to the non-insertable portion of the endoscope.

In at least one embodiment, a surgical instrument is provided that includes a variable length assembly for guiding a flexible endoscopic tool and, in addition, a flexible endoscopic tool. In these embodiments, the variable length assembly includes a first end that is movable relative to a second end wherein the first end is attached to the non-insertable portion of the flexible endoscopic tool.

This Summary is intended to briefly outline certain embodiments of the subject application. It should be understood that the subject application is not limited to the embodiments disclosed in this Summary, and is intended to cover modifications that are within its spirit and scope, as defined by the claims. It should be further understood that this Summary should not be read or construed in a manner that will act to narrow the scope of the claims.

### BRIEF DESCRIPTION OF THE FIGURES

The above-mentioned and other features and advantages of this invention, and the manner of attaining them, will become more apparent and the invention itself will be better understood by reference to the following description of embodiments of the invention taken in conjunction with the accompanying drawings, wherein:

FIG. 1 is a side view of a non-limiting embodiment of an endoscopic system including an endoscopic manipulation accessory connected to an endoscope and a flexible endoscopic tool.

FIG. 2 is a diagrammatical view illustrating a non-limiting embodiment of an endoscope inserted into an overtube and through a patient's mouth and esophagus to perform a surgical activity such as to remove the patient's gall bladder, or perform a cholecystectomy, for example, through a stomach wall.

FIG. 3A is a partial perspective view of the distal portion of the endoscope inserted through the overtube of FIG. 2.

FIG. 3B is a partial perspective view of the distal portion of the endoscope inserted through the overtube of FIG. 2 with an endoscopic tool protruding from a working channel.

FIG. 4 is a perspective view of a non-limiting embodiment of a flexible endoscopic tool.

FIG. 5 is a side view of a non-limiting embodiment of an endoscope with a flexible endoscopic tool inserted therethrough.

FIG. 6 is a perspective view of a non-limiting embodiment of an endoscopic manipulation accessory comprising two telescoping components.

FIG. 7 is another perspective view of the endoscopic manipulation accessory of FIG. 6.

FIG. 8 is a left side view of the endoscopic manipulation accessory of FIG. 6.

FIG. 9 is right side view of the endoscopic manipulation accessory of FIG. 6.

FIG. 10A is a perspective view of the endoscopic manipulation accessory of FIG. 6.

FIG. 10B is an exploded perspective view of the endoscopic manipulation accessory of FIG. 6.

FIG. 11 is a cross-sectional view of the endoscopic manipulation accessory of FIG. 6.

FIGS. 12A-12C illustrate the endoscopic manipulation accessory of FIG. 6 undergoing longitudinal extension and retraction.

FIG. 13 illustrates the endoscopic manipulation accessory of FIG. 6 attached to a working channel port of an endoscope.

FIG. 14A is a side view of a portion of the endoscopic manipulation accessory of FIG. 6 connected to one working channel port of the endoscope of FIG. 13.

FIG. 14B is a cross-sectional view of the portion of the endoscopic manipulation accessory of FIG. 6 attached to the working channel port illustrated in FIG. 14A.

FIG. 15 illustrates a non-limiting embodiment of a flexible endoscopic tool being inserted into an endoscopic manipulation accessory.

FIG. 16 illustrates the flexible endoscopic tool of FIG. 15 being further inserted into the endoscopic manipulation accessory of FIG. 15.

FIG. 17 illustrates the flexible endoscopic tool of FIG. 15 fully inserted into and connected to the endoscopic manipulation accessory of FIG. 15.

FIG. 18 illustrates the connection of the flexible endoscopic tool of FIG. 15 to the endoscopic manipulation accessory of FIG. 15.

FIG. 19 is a cross-sectional view of a non-limiting embodiment of a connector of an endoscopic manipulation accessory.

FIG. 20A is a cross-sectional view of a non-limiting embodiment of an endoscopic manipulation accessory with three telescoping components shown in an extended state.

FIG. 20B is a cross-sectional view of the endoscopic manipulation accessory of FIG. 20A shown in a collapsed or retracted state.

FIG. 21A is a cross-sectional view of portions of two coiled, telescoping components of a non-limiting embodiment of an endoscopic manipulation accessory.

FIG. 21B is a perspective view of the two coiled, telescoping portions illustrated in FIG. 21A.

FIG. 21C is a cross-sectional view of one of the coils forming the two coiled, telescoping components of the variable length assembly of FIG. 21A.

FIG. 22A is a perspective view of a non-limiting embodiment of an endoscopic manipulation accessory including a seal member.

FIG. 22B is an exploded perspective view of the endoscopic manipulation accessory of FIG. 22A.

FIG. 23A is a cross-sectional view of a portion of the endoscopic manipulation accessory of FIG. 22A.

FIG. 23B a cross-sectional view of the portion of the endoscopic manipulation accessory of FIG. 22A attached to a working channel port of an endoscope.

Corresponding reference characters indicate like or corresponding parts throughout the several views. The various illustrated embodiments have been chosen for the convenience of the reader and not to limit the scope of the appended claims.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the various embodiments of the present invention is defined solely by the claims. The features illustrated or described in connection with one exemplary embodiment may be combined with the features of other embodiments. Such modifications and variations are intended to be included within the scope of the present invention.

In the following description, like reference characters designate like or corresponding parts throughout the several views. Also in the following description, it is to be understood that terms such as “forward,” “rearward,” “front,” “back,” “right,” “left,” “upwardly,” “downwardly,” and the like are words of convenience and are not to be construed as limiting terms. The description below is for the purpose of describing various embodiments of the invention and is not intended to limit the invention thereto.

The various embodiments described herein are directed to medical devices and, more particularly, to methods and devices which can be useful in minimally invasive endoscopic procedures carried out with an endoscope and/or a similar surgical instrument. Various embodiments can include methods and devices useful during various medical procedures including, without limitation, methods and devices useful with endoscopes, methods and devices employed through naturally occurring body orifices, and methods and devices related to the guiding of flexible endoscopic tools and assisting a surgeon with the same.

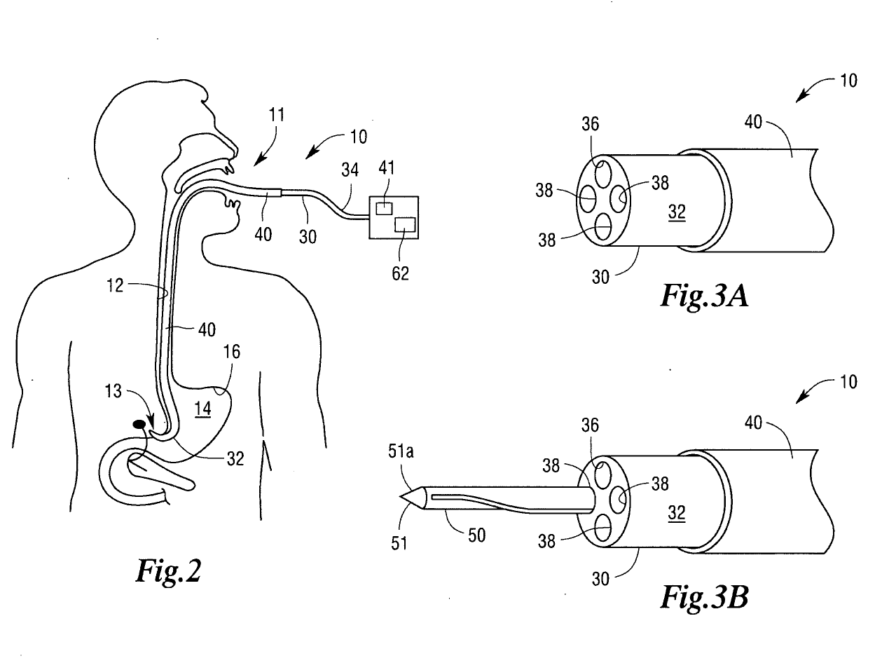

Focusing now on at least one non-limiting embodiment, an endoscopic manipulation accessory can be provided for guiding a flexible surgical tool. In various embodiments, as described in greater detail further below, the length of the endoscopic manipulation accessory can be varied when used in conjunction with the flexible surgical tool and an endoscope. Collectively, the endoscope, the flexible endoscopic tool, and the surgical accessory can comprise an endoscopic system. Various parts and/or uses of an exemplary endoscopic system are set out as follows. FIG. 2 is a diagrammatical view illustrating a non-limiting embodiment of an endoscope 30 inserted into an overtube 40 and inserted through a patient's mouth 11 and esophagus 12 to perform a surgical procedure, such as to remove the patient's gall bladder, or perform a cholecystectomy, for example. In various embodiments, endoscopic system 10 can be inserted through any suitable natural orifice in the patient to form an opening in an organ, or a portion of an organ, such as stomach wall 16, for example. The insertion of the endoscopic system 10 into the patient may occur trans-orally (as depicted in FIG. 2), trans-anally, and/or trans-vaginally, for example. In the example depicted in FIG. 2, the endoscopic system 10 is inserted through the mouth 11 and esophagus 12 of the patient and into the stomach 14 to form an opening 13 through the stomach wall 16.

FIG. 3A is a partial perspective view of the distal portion 32 of the flexible endoscope 30 inserted through the overtube 40 of FIG. 2. A variety of different types of endoscopes are known and, therefore, their specific construction and operation will not be discussed in great detail herein. However, an exemplary, but non-limiting, endoscope and endoscopic system is illustrated and described in U.S. patent application Ser. No. 11/386,861 to Maseda, et al., entitled ENDOSCOPE WORKING CHANNEL WITH MULTIPLE FUNCTIONALITY, the disclosure of which is hereby incorporated by reference in its entirety. In various embodiments, the flexible endoscope 30 has a distal end 32 and a proximal end 34 and may operably support a video camera 36 that communicates with a video display unit 41 that can be viewed by the surgeon during the operation. The flexible endoscope 30 may also comprise one or more working channels 38 extending therethrough for receiving various types of surgical instruments, wherein the working channels 38 may be accessed via working channel ports 62 of the endoscope 30. FIG. 3B is a partial perspective view of the distal portion of the endoscope 30 inserted through the overtube 40 of FIG. 2 with an end effector 51 of an endoscopic tool 50 protruding from a working channel 38. Here, the end effector 51 comprises a hole-forming device 51a. The hole-forming device 51a may comprise, for example, a conventional sphinctorotome, a needle knife and/or other incisor-type instrument that may be inserted through a working channel 38 in the endoscope 30.

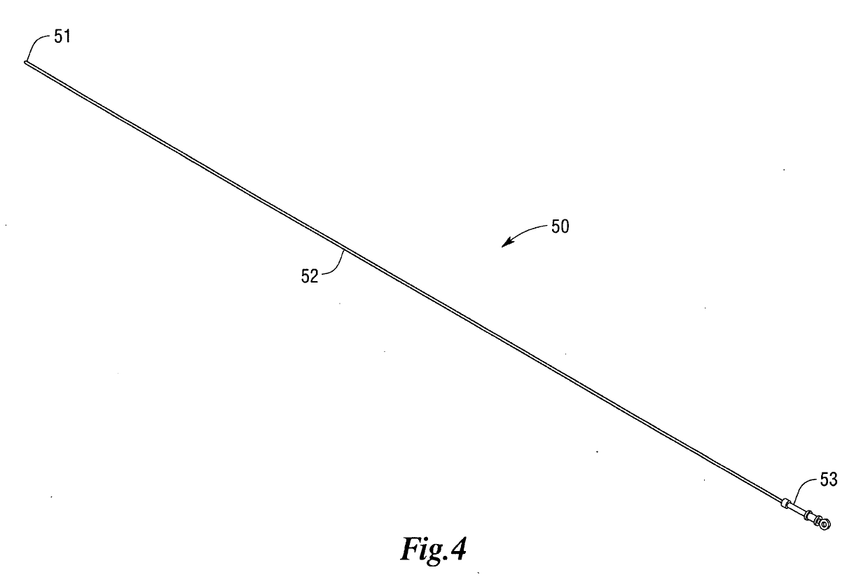

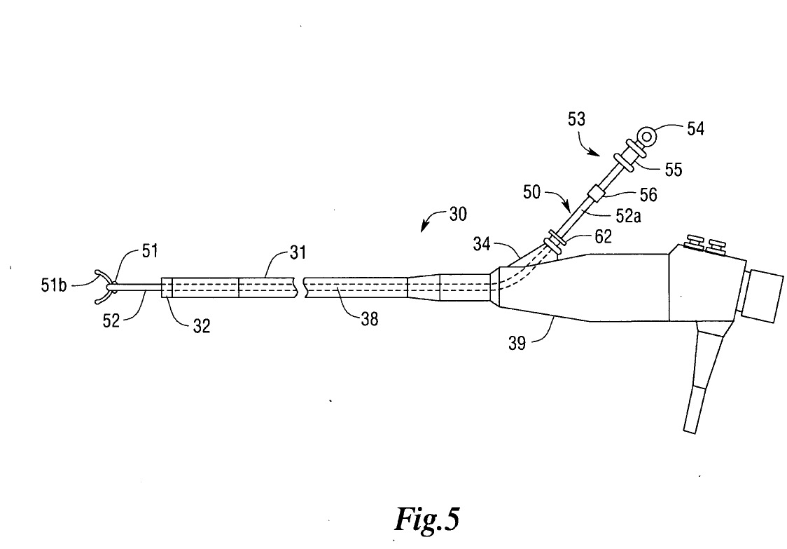

In various embodiments, referring now to FIG. 5, the endoscope 30 can include a proximal handle portion 39 and a distal elongate tubular portion 31, wherein tubular portion 31 can be at least partially flexible and steerable. In at least one embodiment, at least one working channel 38 can be defined within the endoscope 30, wherein the working channel 38 can extend through the handle portion 39 and the elongate tubular portion 31. As indicated above, endoscope 30 can be configured to receive one or more flexible tools therein, such as flexible tool 50, for example, wherein, in at least one embodiment, flexible tool 50 can comprise an end effector 51, a flexible portion 52, and a control member 53 (see, e.g., FIGS. 3A, 3B, 4, and 5), for example. In at least one such embodiment, the working channel 38 of endoscope 30 can be adapted to insertably receive and guide the flexible portion 52 of the flexible endoscopic tool 50 within the endoscope 30. In certain circumstances, two clinicians can operate endoscopic system 10 during a surgical procedure, i.e., an endoscopist, who can steer, articulate, and/or otherwise control the endoscope 30, and a surgeon, who can control the flexible endoscopic tool 50. In such surgical procedures, the surgeon may usually guide the flexible portion 52 (see FIGS. 4 and 5) of surgical tool 50 with one hand while actuating the end effector 51 of flexible tool 50 via control member 53 with his or her other hand as described in greater detail below. In some circumstances, however, pushing on control member 53 of flexible tool 50 may cause at least a portion of the flexible portion 52, especially a portion positioned outside of the endoscope 30, to buckle, thereby making endoscopic system 10 difficult to operate. Such circumstances may arise when a surgeon may be more familiar with rigid surgical tools, such as laparoscopic tools, for example, and less familiar with flexible endoscopic tools. In certain circumstances, a surgeon's lack of familiarity with using a flexible endoscopic tool may be because the operation of a flexible endoscopic tool generally requires the surgeon to use both of his or her hands, whereas the operation of rigid surgical tools may only require the surgeon to use one hand. Furthermore, flexible surgical tools may respond differently than rigid tools when they are pushed through and pulled out of an endoscope.

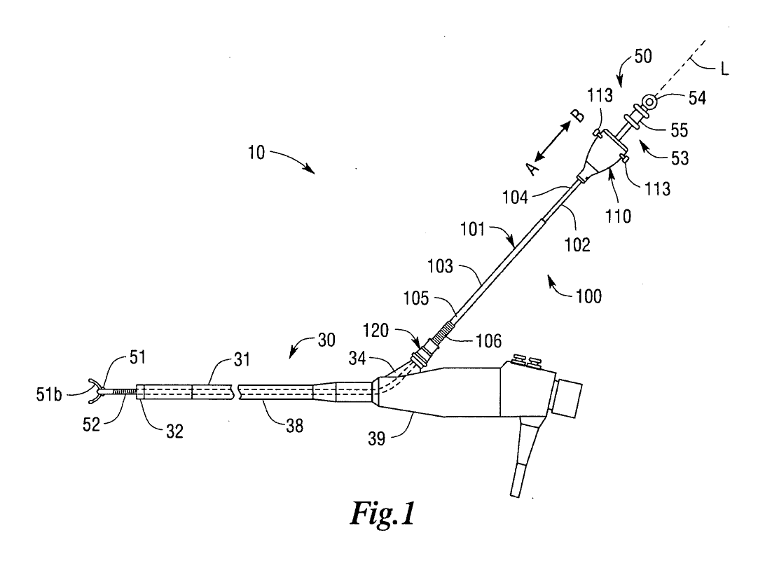

In various embodiments, referring now to FIG. 1, endoscopic system 10 can further include an endoscopic manipulation accessory 100 which can be connected to an endoscope, such as endoscope 30, for example, and/or a flexible endoscopic tool, such as flexible tool 50, for example, wherein endoscopic manipulation accessory 100 can be configured to guide flexible tool 50 into endoscope 30 and support at least a portion of flexible tool 50 as it is moved into and/or out of endoscope 30. In at least one embodiment, endoscopic manipulation accessory 100 can include a variable length assembly 101, a first connector 110, and a second connector 120. Variable length assembly 101 can comprise a first end 104 and a second end 105 that are movable in relation to each other, as described in greater detail below, wherein first connector 110 can be configured to connect the first end 104 of the variable length assembly 101 to the flexible endoscopic tool 50 and second connector 120 can be configured to connect the second end 105 of the variable length assembly 101 to endoscope 30. In various embodiments, variable length assembly 101 can be configured to receive and guide flexible portion 52 of the flexible endoscopic tool 50 and, in certain embodiments, the variable length assembly 101 can be more rigid than the flexible portion 52 of the endoscopic tool 50. In at least one embodiment, the variable length assembly 101 can be substantially rigid in a direction which is transverse to a longitudinal axis L defined by variable length assembly 101. In any event, the variable length assembly 101 can be configured to provide buckling resistance to the flexible portion 52 of the flexible endoscopic tool 50 that is insertably received within an aperture in the variable length assembly 101.

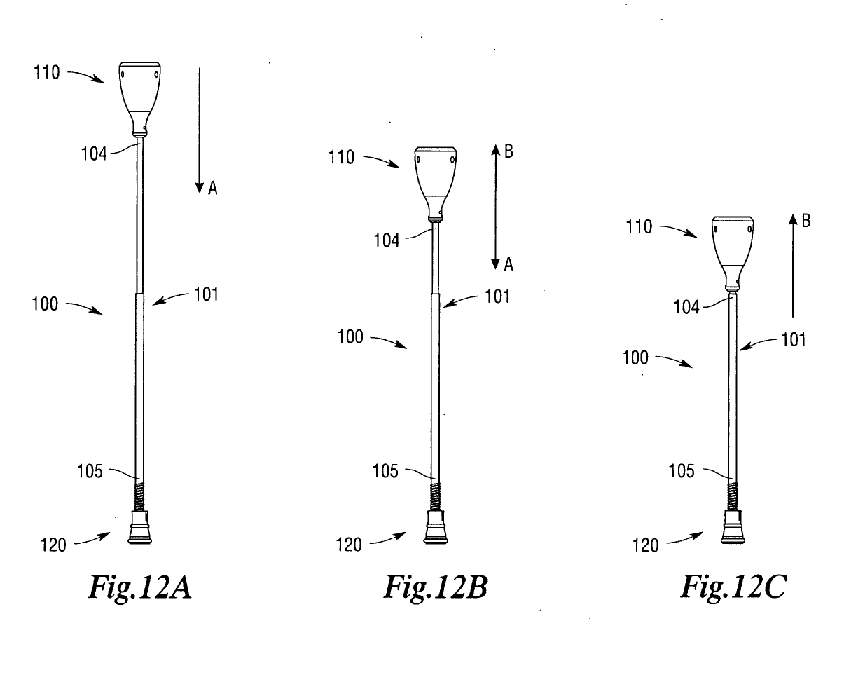

In at least one embodiment, further to the above, variable length assembly 101 of the endoscopic manipulation accessory 100 can comprise a first component 102 and a second component 103, wherein the first and second components 102, 103 are movable relative to one another such that first end 104 is movable relative to second end 105. In various embodiments, referring to FIGS. 12A-12C, such relative movement can be in the directions indicated by arrow A and/or arrow B and along longitudinal axis L. As mentioned above, the variable length assembly 101 can support a flexible endoscopic tool 50 (see FIG. 1) when the tool is passed or inserted through the first and second components 102, 103 such that the first and second components 102, 103 can prevent buckling of the received flexible portion 52 of the endoscopic tool 50. In at least one embodiment, components of the manipulation accessory 100, including, but not limited to, first connector 110, second connector 120, first component 102, and second component 103, may be aligned along longitudinal axis L and may be configured to allow a portion of a flexible endoscopic tool 50 (see FIG. 1) to pass along longitudinal axis L. In various embodiments, the first component 102 can be telescopically engaged with the second component 103. In such embodiments, at least part of the first component 102 is configured to movably slide within the second component 103. Alternatively, the second component 103 may be configured to movably slide within the first component 102. In any event, the variable length assembly 101, including the first and second components 102, 103, may be generally tubular in shape and have a circular cross-section. Other shapes are also contemplated including, but not limited to, shapes having cross-sections with generally square, rectangular, triangular, polygonal, elliptical, and/or any other suitable shape.

In various embodiments, further to the above, the first and second components 102,103 of variable length assembly 101 can be moved between a first, or extended, position and a second, or contracted, position. In various circumstances, further to the above, surgical tool 50 can be mounted to first portion 102 of endoscopic accessory 100 and second portion 103 can be mounted to endoscope 30 such that, when control member 53 of tool 50 is moved toward endoscope 30 to move end effector 51 of tool 50 further into a surgical site, for example, first portion 102 can move relative to second portion 103. Stated another way, first portion 102 can be configured to collapse relative to second portion 103 while still providing relative guided movement between flexible tool 50 and endoscope 30. In at least one such embodiment, the first and second components 102, 103 may be sufficiently rigid to form a system of nested tubes which can prevent, or at least limit, relative lateral movement between the first and second components 102, 103, i.e., prevent movement in a direction which is transverse to longitudinal axis L, for example. As a result of the above, endoscopic manipulation accessory 100 may allow a surgeon to use a flexible endoscopic tool in conjunction with an endoscope such that the flexible endoscopic tool functions in a fashion that is more similar to a rigid laparoscopic instrument than a flexible endoscopic tool and such that the flexible endoscopic tool can be operated with one hand.

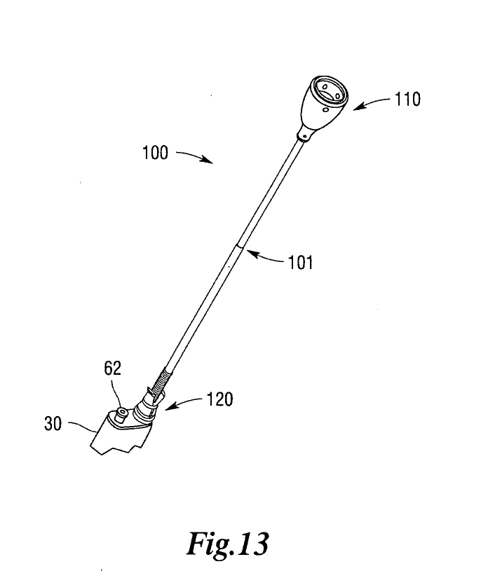

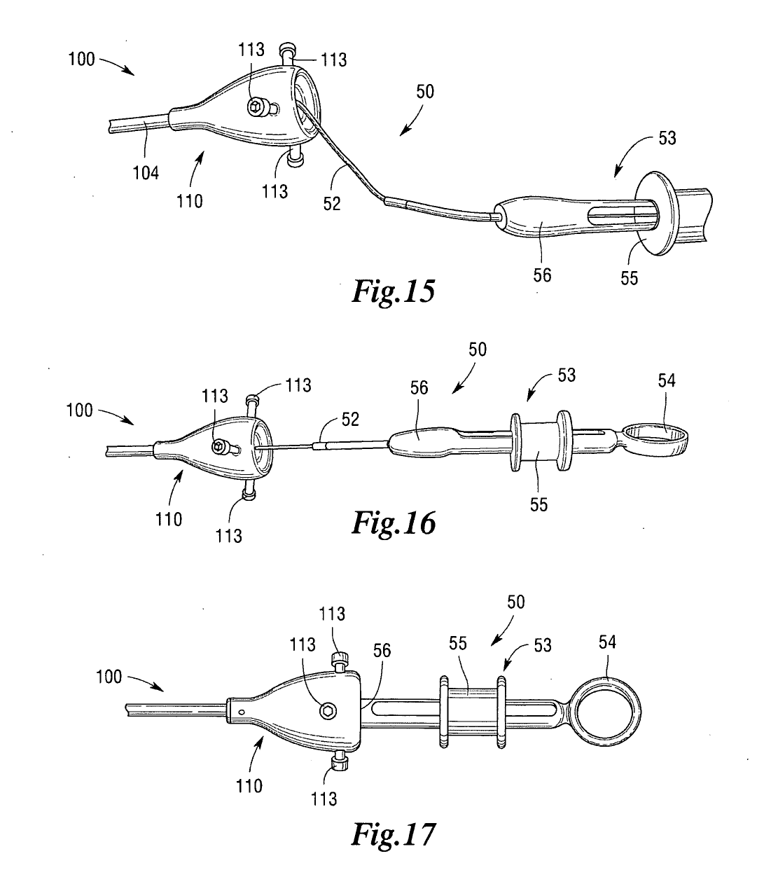

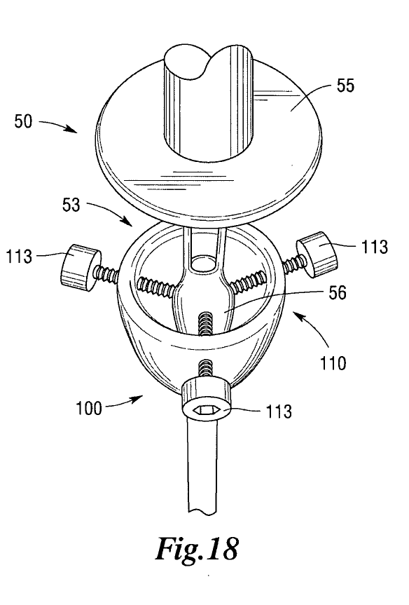

In use, further to the above, endoscope 30 may be inserted through an incision in the patient, or through a trocar inserted into the patient, wherein at least the distal end of the endoscope 30 may be advanced into a surgical site. In certain embodiments, the endoscopic manipulation accessory 100 can then be attached to the endoscope 30 by attaching the second connector 120 of accessory 100 to a working channel port 62 (FIG. 13) on endoscope 30, as described in greater detail further below. In at least one embodiment, the flexible endoscopic tool 50 can then be inserted through accessory 100 and into a channel port 62 of endoscope 30 and then attached to the manipulation accessory 100, as shown in FIGS. 15-18 and also described in greater detail below. In at least one circumstance, the end effector 51 of the flexible endoscopic tool 50 can be fed into the first connector 110 of the manipulation accessory 100 until part of the flexible portion 52 of the endoscopic tool 50 is inserted into the first connector 110 in order to force the end effector 51 through an aperture within the manipulation accessory 100. Thereafter, the flexible endoscopic tool 50 can be fully inserted into the variable length accessory and/or inserted until control member 53 is at least partially positioned within first connector 110. Once positioned therein, as described in greater detail below, control member 53 can be fastened into position. In various alternative embodiments, the manipulation accessory 100 can be attached to endoscope 30 prior to endoscope 30 being inserted into the surgical site. Also, alternatively, flexible endoscopic tool 50 can be attached to the manipulation accessory 100 prior to accessory 100 being attached to endoscope 30.

Once at least a portion of flexible tool 50 has been affixed to first component 102, further to the above, a surgeon may translate or otherwise move the first end 104 of the variable length assembly 101 relative to the second end 105 by applying force to the control member 53 of the flexible endoscopic tool 50 along longitudinal axis L. In at least one embodiment, the surgeon can also apply force to the first component 102 to move first end 104 relative to second end 105. In any event, when a surgeon applies a pushing force from first end 104 toward second end 105, the manipulation accessory 100, including variable length assembly 101, can collapse or shorten in the direction of arrow A. When a surgeon applies a pulling force in a direction opposite to the pushing force, the manipulation accessory 100, including variable length assembly 101, can extend, or lengthen, in the direction of arrow B. In such embodiments, a surgeon, who is already familiar with laparoscopic procedures, may feel more comfortable using a flexible endoscopic tool 50 because of the guidance and enhanced rigidity provided by endoscopic manipulation accessory 100. Additionally, a surgeon is capable of moving and/or operating the flexible endoscopic tool 50 with only one hand if desired, thus freeing their other hand.

In various embodiments, further to the above, the range of movement between the first component 102 and the second component 103 can be limited such that the maximum extended position and the maximum contracted position of variable length assembly 101 can be positively defined. In at least one embodiment, referring generally to FIG. 9, first component 102 can further include at least one stop pin 109 extending therefrom which can be configured to be received within a second slot 108 of the second component 103, wherein movement of the first component 102 with respect to the second component 103 can cause the stop pin 109 to move within second slot 108. Accordingly, the relative movement between the first and second components 102, 103, and the relative movement between the first and second ends 104, 105 of the variable length assembly 101, can be restrained when stop pin 109 contacts either end of slot 108 within second component 103. Stated another way, the ends of slot 108 can define the maximum stroke length of first component 102 relative to second component 103. In various embodiments, first component 102 can comprise at least one first slot 107 and, in addition, second component 103 can comprise at least one stop pin (not illustrated) extending therefrom which can be configured to be received within a first slot 107 such that, similar to the above, the ends of first slot 107 can define the stroke length of the relative movement between first component 102 and second component 103.

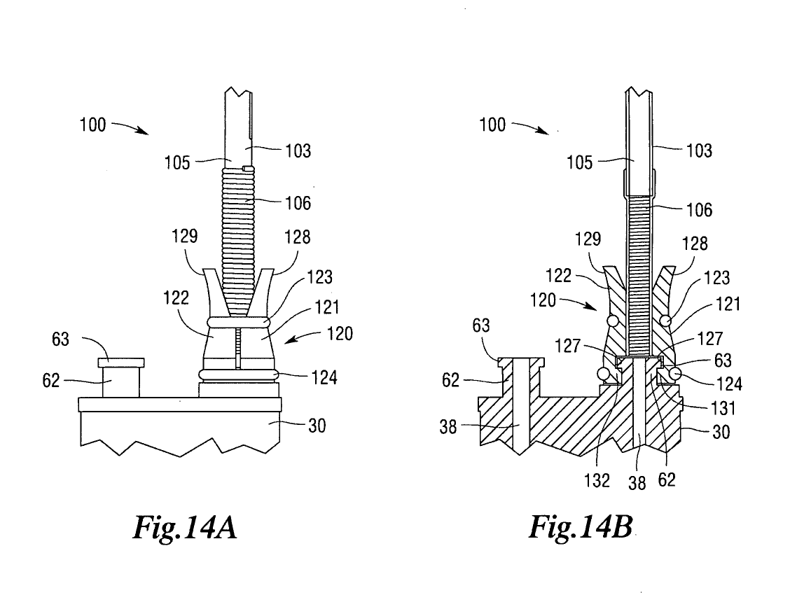

In various embodiments, as described above, manipulation accessory 100 can be attached to endoscope 30 via second connector 120. More particularly, in at least one embodiment, second connector 120 can be mounted to a flange, or another suitable retaining member, extending from endoscope 30 such that the aperture extending through manipulation accessory 100 can be aligned, or at least substantially aligned, with a working channel port 62 in endoscope 30. In certain embodiments, referring to FIG. 13, an endoscope can comprise two or more working channel ports, wherein two or more manipulation accessories 100, for example, can be mounted to the working channel ports 62 such that two or more flexible endoscopic tools can be used independently of and/or simultaneously with one another. FIG. 14A is a side view, and FIG. 14B is a cross-sectional view, of the second connector 120 mounted to a working channel port 62 of the endoscope 30. More particularly, referring to FIGS. 14A and 14B, the second connector 120 can be clipped to a flange 63 surrounding, or at least partially surrounding, working channel port 62. In at least the illustrated embodiment, the second connector 120 can further comprise a working channel recess 127 configured to receive the flange 63 such that the flange 63 is enveloped, or at least partially enveloped, by the second connector 120. In at least one embodiment, the second connector 120 can comprise one or more clip pieces, such as pivot clip pieces 121 and 122, for example, which can be pivoted, rotated, and/or otherwise suitably moved between latched (FIGS. 14A and 14B) and unlatched configurations to engage and disengage second connector 120.

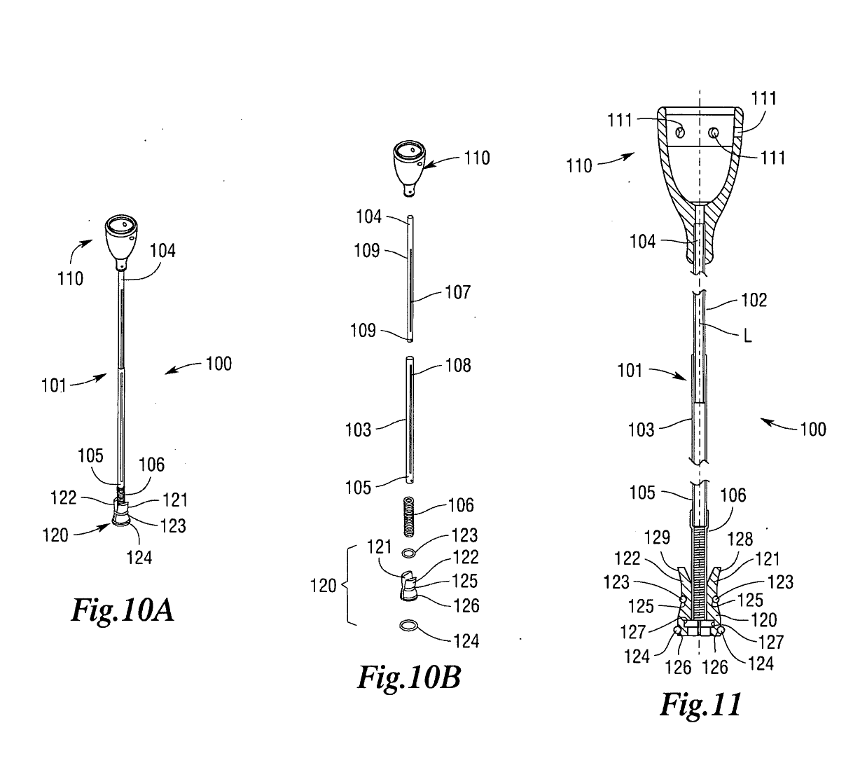

In various embodiments, further to the above, pivot clip pieces 121 and 122 can comprise finger grip portions 128 and 129, respectively, wherein finger grip portions 128, 129 can be moved toward each other in order to move lock portions 131 and 132 of pivot clip pieces 121 and 122, respectively, away from each other and, in addition, away from the lock flange 63. Once lock portions 131 and 132 have been sufficiently disengaged from flange 63, second connector 120 can be pulled away from flange 63 along longitudinal axis L, for example, in order to disengage second connector 120 from the working channel port 62. In at least one embodiment, referring again to FIGS. 10A, 10B, 14A and 14B, the first clip piece 121 and the second clip piece 122 can be held together by a first O-ring 123 and a second O-ring 124, wherein O-ring 123 can sit within a groove 125 and O-ring 124 can sit within a groove 126 formed within clip pieces 121 and 122. Each O-ring 123, 124 can be comprised of an elastomeric material(s) such as natural rubber, synthetic rubber, latex, elastomers, elastic polymers, and/or any other suitable material, for example, wherein, O-rings 123 and 124 can be configured to bias clip pieces 121 and 122 into their closed positions. In order to engage second connector 120 with the working channel port 62, second connector 120 can be placed against the lock flange 63 such that an axial force applied to second connector 120 can cause lock portions 131 and 132 to displace outwardly. Once displaced outwardly, lock portions 131 and 132 can be pushed back towards each other and towards working channel port 62 by O-rings 123, 124 until they are permitted to resiliently move behind lock flange 63 and, accordingly, seat second connector 120 to the working channel port 62. In certain other embodiments, further to the above, a user can concurrently apply forces to finger portions 128, 129 such that at least a portion of the each clip piece 121, 122 can abut the outer surface of second component 103 and/or strain relief mechanism 106 (described in greater detail below), for example, and rotate about an axis on the surface thereof in order to move lock portions 131 and 132 away from lock flange 63. In such circumstances, lock portions 131 and 132 can expand O-ring 123 and/or O-ring 124. Once clip pieces 121 and 122 have been suitably aligned with lock flange 63, finger grip portions 128 and 129 can be released in order to permit O-rings 123 and 124 to resiliently contract and bias clip pieces 121 and 122 into engagement with lock flange 63.

In various embodiments, as outlined above, variable length assembly 101 can comprise a rigid assembly wherein, in at least one embodiment, the manipulation accessory 100 may not be rotatable relative to endoscope 30 and/or movable in directions which are transverse to longitudinal axis L. In other various embodiments, however, the variable length assembly 101 may further comprise a strain relief mechanism, such as strain relief mechanism 106, for example, which can be configured to permit at least a portion of manipulation accessory 100 to rotate or articulate relative to endoscope 30 and/or translate transversely relative to longitudinal axis L, for example. In at least one embodiment, strain relief mechanism 106 can connect, or attach, the second component 103 to second connector 120. In various embodiments, strain relief mechanism 106 can include a spring, an elastic member, and/or other any other suitable structure or material that allows second component 103 to pivot relative to endoscope 30 and/or otherwise move relative to longitudinal axis L. More particularly, in at least one such embodiment, the strain relief mechanism 106 can be configured to permit second component 103 to articulate with respect to the second connector 120 and, in certain embodiments, strain relief mechanism 106 can be configured to resiliently bend to accommodate such relative movement. In such embodiments, strain relief mechanism 106 can relieve strain that would otherwise be experienced between second component 103 and second connector 120 when a transverse bending force is applied to the endoscopic manipulation accessory 100, for example. In at least one embodiment, strain relief mechanism 106 can comprise an elastic sleeve which may be stretched over at least a portion of the second component 103 and/or second connector 120 such that it is securely attached thereto. In at least one embodiment, referring to FIG. 14B, at least a portion of strain relief mechanism 106 can be captured and/or press-fit within an aperture in second connector 120. In certain embodiments, O-rings 123 and 124 can be configured to apply a biasing force to first and second pivot clips 121, 122 such that pivot clips 121 and 22 contact strain relief mechanism 106 with enough force to form a secure connection and retain strain relief mechanism 106 therebetween. While the second connector 120 is shown in the illustrated embodiment as a separate component which is assembled to the variable length assembly 101, the second connector 120 may, alternatively, be unitarily or integrally formed with the variable length assembly 101 and/or the strain relief mechanism 106. In certain embodiments, although not illustrated, the strain relief mechanism 106 may be unitarily or integrally formed with variable length assembly 101.

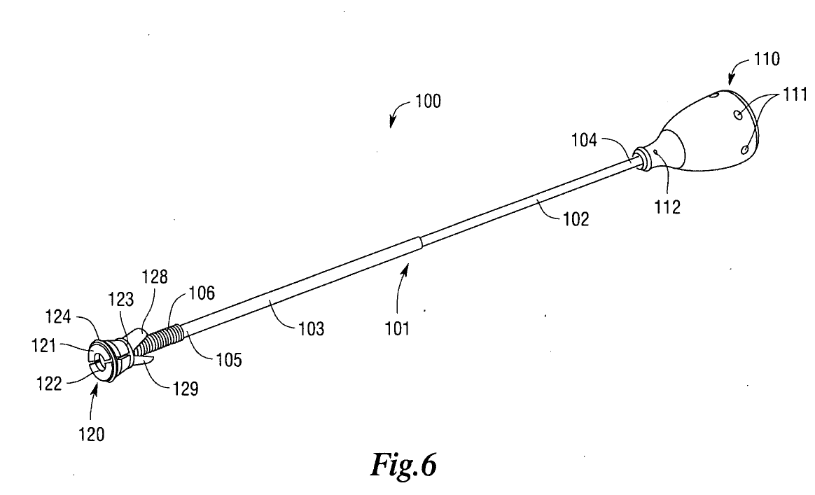

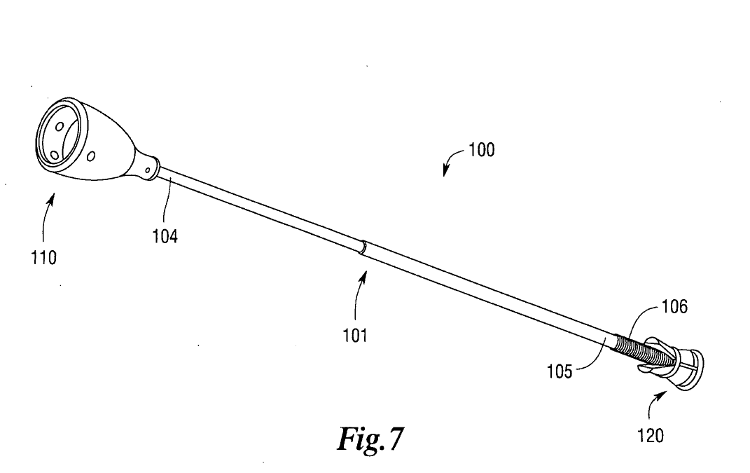

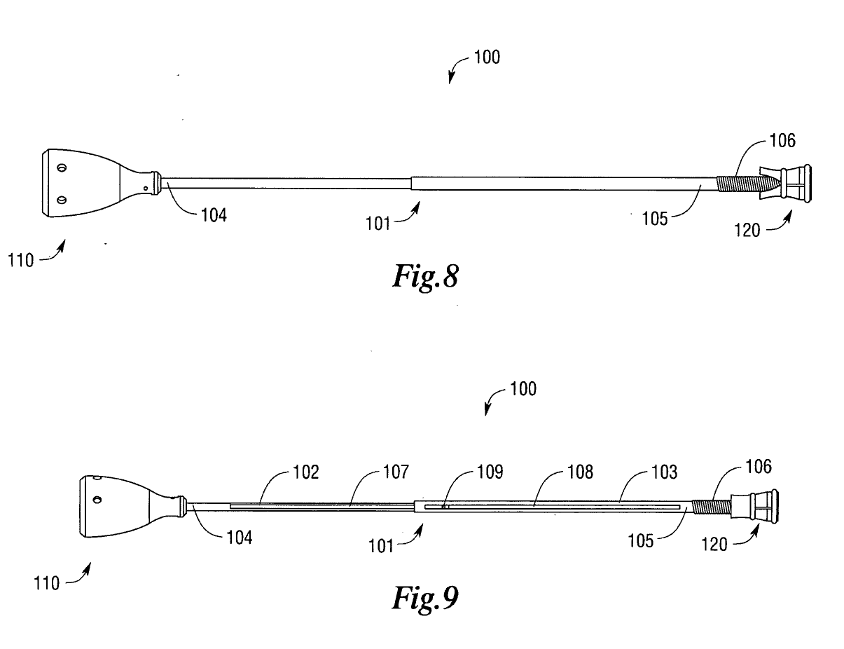

In various embodiments, as discussed above, a flexible surgical tool, such as flexible endoscopic tool 50, for example, can be releasably mounted, or connected, to manipulation assembly 100 via first connector 110. In at least one embodiment, referring to FIGS. 1 and 15, first connector 110 can comprise fasteners 113 which can be configured to engage a first, or stationary, portion of the flexible endoscopic tool 50, such as spool stop 56, for example. In at least one embodiment, fasteners 113 can comprise screws that are tightened against spool stop 56 so as to hold the stationary portion of flexible endoscopic tool 50 relatively still with respect to the first connector 110. In at least one such embodiment, referring primarily to FIGS. 6 and 11, first connector 110 can further comprise at least one fastener hole 111 which can be configured to threadably receive at least a portion of a fastener 113 therein. In certain embodiments, fastener holes 111 can include pre-formed threads which can be threadably engaged by fasteners 113 while, in other embodiments, fasteners 113 can be configured to form threads within fastener holes 111. While fasteners 113 can comprise screws as described above, fasteners 113 can include any appropriate fastener that is capable of clamping, securing, locking, or otherwise coupling first connector 110 to part of the flexible endoscopic tool 50, including, but not limited to thumb screws, bolts, clamps, and the like.

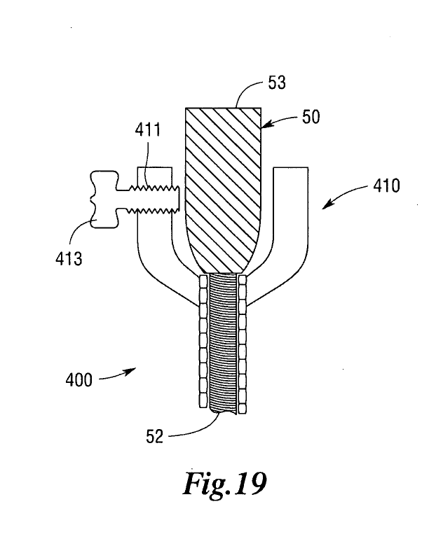

A connector of an alternative endoscopic manipulation accessory is illustrated in FIG. 19. In various embodiments, connector 410 of endoscopic manipulation accessory 400, similar to connector 110 of endoscopic manipulation accessory 100, can be configured to receive at least a portion of a flexible surgical tool, such as surgical tool 50, for example, therein. In various embodiments, the first connector 410 can include a fastener hole 411 and a fastener 413 positioned therein, wherein fastener 413 comprises a thumbscrew. In FIG. 19, fastener 413 is shown disengaged from control member 53 of the flexible endoscopic tool 50. In use, turning fastener 413 a number of turns can move fastener 413 into contact with the control member 53 such that the flexible endoscopic tool 50 is secured to first connector 410.

In various embodiments, referring to FIGS. 10A and 10B, first connector 110 can comprise a separate component which is attached to first component 102 of the variable length assembly 101. In at least one embodiment, referring to FIG. 11, first connector 110 can comprise an aperture 115 which can be configured to receive the first end 104 of the variable length assembly 101, wherein, in certain embodiments, the first end 104 can be press-fit and/or snap-fit within aperture 115. In at least one embodiment, an adhesive, such as an epoxy, for example, can be utilized to retain first end 104 within aperture 115. In certain embodiments, first connector 110 may include at least one set pin (not illustrated) and at least one set pin hole 112 (FIG. 6) adapted to receive a set pin, wherein the set pin can be configured to engage the first end 104 of the variable length assembly 101 in order to attach first connector 110 to first component 102 at or near first end 104 of the variable length assembly 101. While the first connector 110 is illustrated as a separate component in FIGS. 10A and 10B, the first connector 110 may be, although not illustrated, unitarily formed with and/or integrally connected to the variable length assembly 101. In at least one such embodiment, the first component 102 and the first connector 110 can be formed from one continuous piece of material. In any event, once at least a portion of flexible endoscopic tool 50 has been secured to first connector 110, tool 50 can be operated as otherwise described herein.

In various embodiments, as described above, a flexible endoscopic tool, such as endoscopic tool 50, for example, can include an end effector, such as end effector 51, for example, that is operable, controlled, and/or otherwise adjusted when a user manipulates control member 53 of endoscopic tool 50. More particularly, as also outlined above and referring to FIGS. 15-18, endoscopic tool 50 can comprise a stationary portion, such as spool stop 56, and a movable portion, such as displaceable spool 55, wherein relative movement between displaceable spool 55 and spool stop 56 can actuate end effector 51. In at least one such embodiment, although not illustrated, displaceable spool 55 can be operably engaged with an actuator that extends between displaceable spool 55 and end effector 51 wherein the actuator can be configured to slide within an outer sheath of flexible portion 52 which can be seen in FIG. 4. In various embodiments, referring again to FIG. 1, the end effector 51 can comprise a grasper or biopsy forceps 51b wherein movement of the displaceable spool 55 relative to spool stop 56 can cause the grasper or forceps 51b to selectively open and close. For example, when displaceable spool 55 is pulled proximally, i.e., away from first connector 110, the actuator attached to displaceable spool 55 can close, or at least partially close, the jaws of end effector 51 whereas, when displaceable spool 55 is pushed distally, i.e., toward first connector 110, the actuator attached to displaceable spool 55 can open, or at least partially open, the jaws of end effector 51. In various embodiments, the actuator operably engaged with displaceable spool 55 can comprise a wire or cord, wherein the wire or cord can be sufficiently rigid such that it can transmit the pushing and pulling forces applied thereto to end effector 51. In at least one such embodiment, the outer sheath of flexible portion 52 can guide and/or support the actuator when it slides therein.

In various circumstances, however, the outer sheath of flexible portion 52 may be unable to properly support the movable actuator positioned therein. In at least one circumstance, as discussed above and referring again to FIG. 5, the flexible portion 52 of endoscopic tool 50, especially portion 52a which is positioned outside of endoscope 30, may bend and/or otherwise suitably move when displaceable spool 55 is displaced relative to stop spool 56, thereby inhibiting the operation of flexible endoscopic tool 50. In various embodiments, as discussed above and referring again to FIG. 1, the endoscopic system 10 can comprise an endoscopic manipulation accessory 100 that is coupled to both endoscope 30 and flexible endoscopic tool 50 which can be configured to guide and support flexible portion 52 of surgical tool 50, especially the outer sheath of flexible portion 52 supporting the actuator. In such embodiments, the surgeon can move end effector 51 further into a surgical site by pushing stop spool 56 and first connector 110 toward endoscope 30, wherein the surgeon can at least partially withdraw end effector 51 from the surgical site by pulling on stop spool 56 and first connector 110. Further to the above, in various embodiments, the surgeon operate the end effector 51 of the surgical tool 50 by moving movable spool 55 relative to stop spool 56 in proximal and distal directions as described above. In various circumstances, the surgeon can move and operate an end effector sequentially and, in other circumstances, the surgeon can perform these tasks simultaneously.

During an endoscopic surgical procedure, it may be desirable to insufflate, or deliver gas (e.g. carbon dioxide), to a body cavity of a patient such that the cavity can be enlarged and the operating space within the cavity can be increased. Into this insufflated cavity, an endoscope, such as endoscope 30, for example, may be inserted to perform the endoscopic surgical procedure with a flexible endoscopic tool, such as flexible endoscopic tool 50 (see FIG. 4), for example, as described above. As also described above, an endoscopic manipulation accessory 100 may be connected to endoscope 30 via a working channel port 62 and, in addition, flexible endoscopic tool 50 may be inserted through the manipulation accessory 100, through the endoscope 30 via a working channel 38, and into the patient's insufflated cavity (see FIGS. 1, 2, and 5). During such a procedure, however, insufflation gas may escape from the body cavity through the working channel 38 of the endoscope 30 and out port 62, thus reducing gas pressure within the body cavity and possibly decreasing the operating space for the procedure. Various embodiments are described below which can be configured to resist or prevent the gas from escaping during the endoscopic surgical procedure.

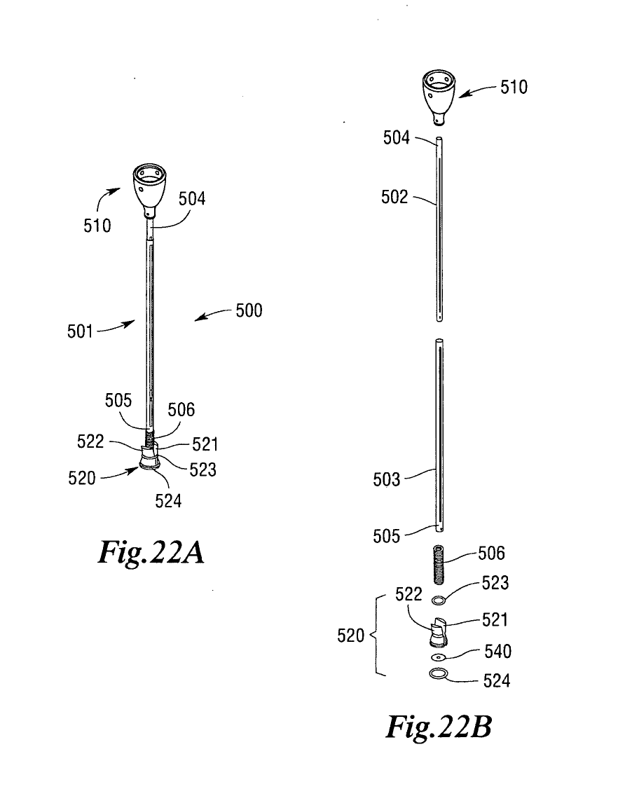

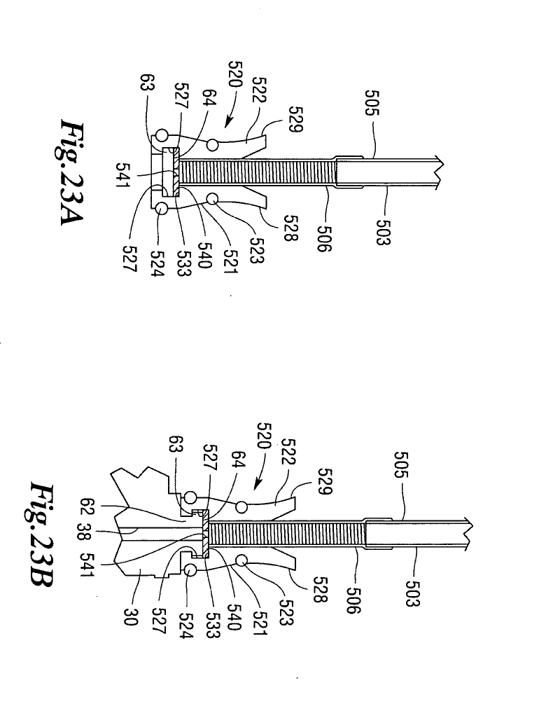

In various embodiments further to the above, an endoscopic manipulation accessory, such as endoscopic manipulation accessory 500 (FIGS. 22A-23B), for example, may further include a seal member such as seal member 540, for example, wherein the seal member can serve to form an airtight or air-resistant barrier between working channel port 62 (FIG. 23B) and the external environment. In certain embodiments, seal member 540 can comprise at least one groove or aperture configured to receive at least one surgical tool such as flexible endoscopic tool 50, for example, when the tool 50 is passed through endoscopic manipulation accessory 500. In at least one embodiment, flexible endoscopic tool 50 may pass through seal member 540 via passageway 541 defined therein, such at least a portion of the perimeter of passageway 541is in contact with the tool 50 such that gas can be prevented or resisted from flowing out of the patient's body cavity via the working channel 38 and port 62 of endoscope 30 to the outside environment and vice versa. For the purposes of clarity, the seal member 540 is shown with hatching in FIGS. 23A and 23B while the other portions of the second connector 520 are shown without hatching.

Referring now to FIGS. 22A-22B, endoscopic manipulation accessory 500 may be similar to manipulation accessory 100 in that manipulation accessory 500 is configured to guide a flexible endoscopic tool. Manipulation accessory 500 includes a variable length assembly 501 that includes first component 502 telescopically engaged with second component 503 such that a first end 504 of the variable length assembly 501 is movable relative to a second end 505. Similar to the above, the first and second components 502, 503 are adapted to receive a flexible portion of the endoscopic tool. Also, the endoscopic manipulation accessory 500 includes a first connector 510 and a second connector 520. The first connector 510 is configured to releasably connect the first component 502 to the flexible endoscopic tool 50 and the second connector 520 is configured to releasably connect the second component 503 to an endoscope, as outlined above.

Similar to second connector 120, described above, second connector 520 may be configured to engage at least a portion of working channel port 62 of endoscope 30 (see FIG. 23B). In such embodiments, second connector 520 may include first clip piece 521 and second clip piece 522 which can be held together by a first O-ring 523 and a second O-ring 524, wherein O-ring 523 can sit within a groove 525 and O-ring 524 can sit within a groove 526. As explained above with respect to second connector 120, O-rings, such as O-rings 523 and 524 can be configured to bias clip pieces 521 and 522 into their closed positions. Also similar to the above, the second connector 520 can comprise a working channel recess 527 configured to receive the flange 63 of working channel port 62 such that the flange 63 may be enveloped, or at least partially enveloped, by the second connector 520.

In various embodiments, seal member 540 may be attached to an end of strain relief mechanism 506 such that, when second connector 520 engages a working channel port 62, the seal member 540 may be compressed between surface 533 of clip pieces 521, 522 and surface 64 of working channel port 62. In at least one such embodiment, a sealing interface can exist between surface 533 and seal member 540 and, in addition, between seal member 540 and channel port surface 64. In other words, the seal member may be “sandwiched” between the clip pieces 521 and 522 and the working channel port 62. In certain embodiments, the seal member 540 can be made from an elastic, compressible and resilient material or materials such as, for example, silicone, rubber, elastomers, and/or any suitable combination thereof. Alternatively, the seal member 540 may be attached to a portion of clip pieces 521 and 522, such as at surface 533, for example. Such attachment may be made by using an adhesive and/or ultrasonic welding to connect the seal member 540 to the clip pieces 521 and 522. In such embodiments, the seal member 540 may stretch when the clip pieces 521 and 522 are pivoted away from one another, and seal member 540 may resiliently return to its illustrated shape when clip pieces 521 and 522 are returned to their illustrated positions.

In use during a surgical procedure, after inserting an endoscope 30 into a patient, as described above, the endoscopic manipulation accessory 500 may be connected to the endoscope 30 via second connector 520 at working channel port 62, as shown in FIG. 23B. In various embodiments, second connector 520 may be engaged with port 62 such that at least a portion of seal 540 engages port 62. In at least one such embodiment, a face surface of seal 540 can be configured to abut flange 63 such that a sealing interface is created therebetween. The second connector 520 can be sufficiently connected to port 62 such that at least a portion of seal 540 is biased against surface 63. Next, in at least one embodiment, a flexible endoscopic tool 50 may be inserted through the endoscopic manipulation accessory 500 such that the tool 50 passes through seal member 540 via passageway 541, thereby creating an airtight or air-resistant barrier between the working channel port 62 and the external environment. Owing to the seal member 540, insufflation gas supplied to a surgical site can be prevented or hindered from escaping from the patient, thus maintaining insufflation of the patient's internal cavity. Finally, the surgical procedure may be performed using the flexible endoscopic tool 50 within the insufflated cavity. In certain embodiments, an insufflation gas can be supplied to the surgical site via a Veress needle and/or endoscope 30 and/or overtube 40 (see FIG. 2).

Further to the above, while the grasper jaws of end effector 51, illustrated in FIG. 1, can be entirely suitable for their intended purpose, other end effectors are contemplated including, but not limited to, a specimen retrieval bag, biopsy jaws with a spike, a snare loop, scissors, and/or a hook knife, for example. Various end effectors are described in greater detail in commonly-owned U.S. patent application Ser. No. 12/133,109 to Zwolinski et al., entitled ENDOSCOPIC DROP OFF BAG; U.S. patent application Ser. No. 11/610,803 to Nobis et al., entitled MANUALLY ARTICULATING DEVICES; and U.S. patent application Ser. No. 12/133,953 to Nobis et al., entitled MANUALLY ARTICULATING DEVICES, the disclosures of which are incorporated by reference in their entirety.

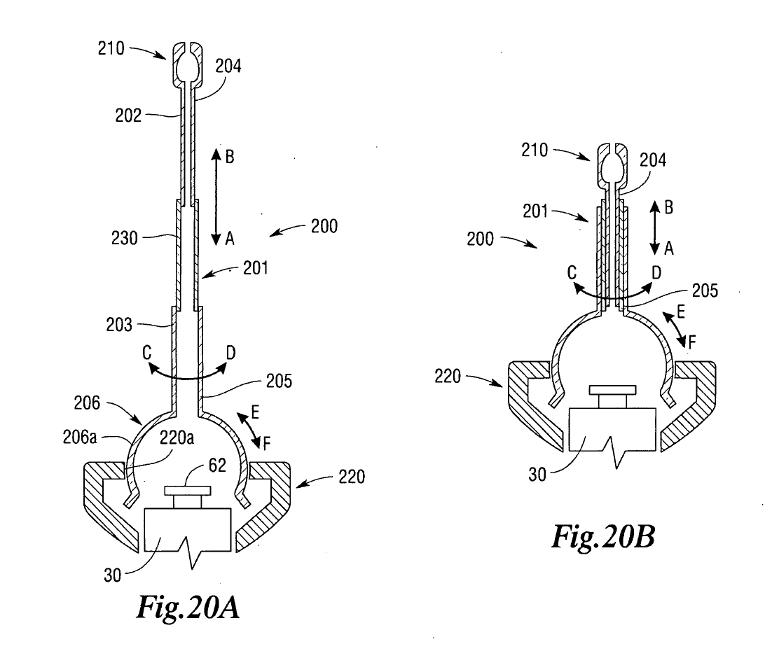

In various embodiments, as described above, an endoscopic manipulation accessory can comprise two or more relatively-moving or telescoping components. In at least one embodiment, referring to FIG. 20A, an endoscopic manipulation accessory 200 can include a variable length assembly 201 comprising three telescoping components which are shown in an extended state. FIG. 20B illustrates the endoscopic manipulation accessory 200 in a collapsed or retracted state. In various embodiments, the manipulation accessory 200 can include three components, i.e., a first component 202, a second component 203, and an intermediate component 230, that are telescopically engaged with each other and can comprise variable length assembly 201. In certain embodiments, first component 202 can be slideably received in intermediate component 230 and, in addition, intermediate component 230 can be slideably received in second component 203. In at least one embodiment, manipulation accessory 200 can further comprise a first connector 210 and a second connector 220, wherein first connector 210 can be similar to first connector 110 and can connect tool 50 to endoscopic manipulation accessory 200, and wherein second connector 220 can be similar to second connector 120 and can be configured to attach to endoscopic manipulation accessory 200 to endoscope 30. Also similar to the above, the variable length assembly 201 can further comprise a first end 204 and a second end 205 which can be movable relative to one another.

In various embodiments, referring again to FIGS. 20A and 20B, components 202, 203, and/or 230 can be moved in directions indicated by arrows A and B in order to collapse and expand variable length assembly 201, respectively. In at least one embodiment, some of the components 202, 203 and/or 230 can be selectively moved in directions A and/or B while other components can remain stationary. In certain embodiments, some of components 202, 203 and 230 can be moved in direction A while others can be moved in direction B. In any event, the manipulation accessory 200 can further include strain relief mechanism 206 which, in at least one embodiment, can comprise a ball 206a that is received within a socket 220a of second connector 220. In at least one such embodiment, such a ball-and-socket arrangement can allow variable length assembly 201, for example, to be rotated about a longitudinal axis as indicated by arrows C and D and/or pivoted in various different directions as indicated by arrows E and F. In various embodiments, second connector 220 can comprise one or more stops which can be configured to limit the relative rotation between ball 206a and second connector 220 to a defined range of motion.

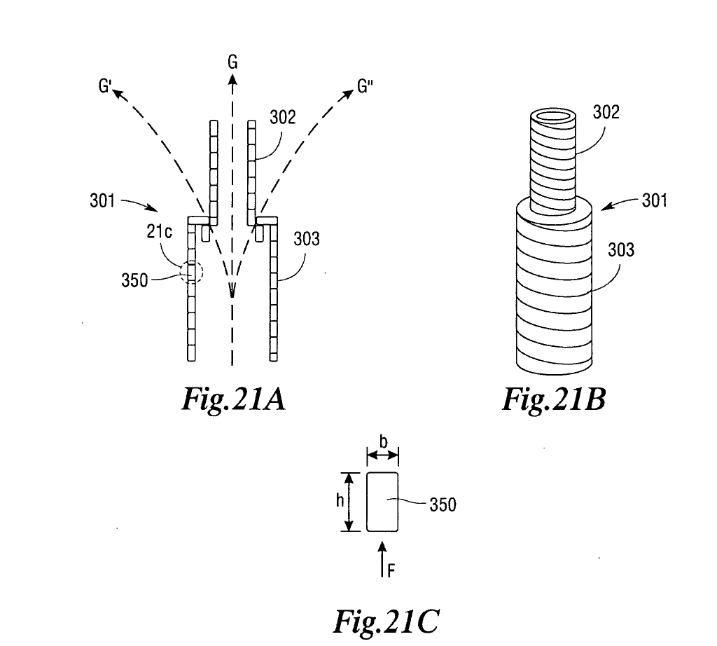

In various embodiments, as described above, the telescoping components of an endoscopic manipulation accessory may be substantially rigid. In various embodiments, however, the telescoping components, or at least portions of the telescoping components, may be semi-rigid and bendable such that they can elastically return to their original form without substantial permanent deformation. For example, the telescoping components 102, 103 and/or the telescoping components 202, 203, 230, as described above, may be made of an elastomeric material, such as rubber, for example. In certain embodiments, a coiled material may be used to form the telescoping components of a variable length assembly. In various embodiments, referring now to FIGS. 21A-21C, variable length assembly 301 can comprise a first component 302 and a second component 303, wherein each component can be comprised of a coiled material 350. In at least one embodiment, coiled material 350 can comprise a rectangular cross-sectional shape having a height h and a width b that are selected such that the cross-sectional moment of inertia of the rectangular cross-sectional shape provides sufficient radial stiffness to prevent, or at least inhibit, the buckling of a flexible endoscopic instrument passing through the variable length assembly 301. The moment of inertia (I) for a rectangular cross-section can be defined as:

$$I = \frac{1}{12} {bh}^{3}$$

Owing to the exponent associated with the height h, the moment of inertia l is most efficiently increased by making height h large compared to width b. In at least one embodiment, the height h and the width b of the cross-sectional shape can also be chosen to allow the variable length assembly 301 to bend in directions represented by arrows G′ and G″, for example. In other words, the height h and the width b can be selected such that there is enhanced longitudinal rigidity in the direction of arrow G but decreased radial rigidity in a direction transverse to the direction of arrow G. In any event, in at least one embodiment, variable length assembly 301 can be used in a manner similar to the variable length assembly 101, for example, in that the length of variable length assembly 301 can be varied to move a flexible surgical instrument within a surgical site and in that the variable length assembly 301 can guide the surgical instrument.

In various embodiments, the telescoping components of a variable length assembly, such as components 102, 103 and/or components 202, 203, 230, may be manufactured from a rigid material which is formed into a tube and then cut with a laser to remove material in a pattern that results in an at least partially flexible tube. In at least one such embodiment, such a laser cut tube may be created by providing a tube, made, for instance, of a metal, and then rotating the tube with respect to a laser or otherwise passing a laser around the tube to cut patterns into the exterior and/or interior of the tube with the laser. The patterns may be micro-sized, may extend through a wall of the tube, and/or may be helical in shape along a longitudinal axis of the tube. In any event, the resulting laser cut tube may be capable of bending without permanently deforming. Such a laser cut tube may have advantages over nested coil tubes described above and illustrated in FIGS. 21A-21C in that a laser cut tube may be less likely to pull apart, or become disassembled, like a coil based tube may. Also, a laser cut tube may have a thinner wall compared to a coil based tube which may lead to smaller cross-sectional areas and better outer diameter-to-inner diameter ratios.

The devices disclosed herein can be designed to be disposed of after a single use, or they can be designed to be used multiple times. In either case, however, a device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present disclosure and appended claims.

Preferably, the various embodiments described herein will be processed before surgery. First, a new or used instrument is obtained and if necessary cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that the device is sterilized. This can be done by any number of ways known to those skilled in the art including beta or gamma radiation, ethylene oxide, and/or steam.

Although various embodiments have been described herein, many modifications and variations to those embodiments may be implemented. The variable length assembly may be integral and unitary with the endoscope. Alternatively, the variable length assembly may be integral and unitary with the flexible endoscopic tool. Also alternatively, the variable length assembly may be unitary and integral with both the endoscope and the flexible endoscopic tool. In other words, instead of begin releasably attached to both the endoscope and the flexible endoscopic tool, the variable length assembly may be non-releasably or permanently attached to the endoscope and/or the flexible endoscopic tool.

Further, the variable length assembly may also include a single component. In such an embodiment, variable length assembly may have its length varied by being constructed of a corrugated elongate shaft, for example a shaft made from a plastic tube that is accordion-like in function. Alternatively, the variable length assembly may have its length varied by being constructed of a spring made from a coiled wire. Also, where materials are disclosed for certain components, other materials may be used. The foregoing description and following claims are intended to convey and cover all such modification and variations.

Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A surgical accessory for guiding a flexible endoscopic tool, said surgical accessory comprising:
a variable length assembly comprising a first component and a second component, wherein said first component is telescopically engaged with said second component, and wherein said first component and said second component are adapted to receive a flexible portion of the flexible endoscopic tool;
a first connector configured to releasably connect said first component to the flexible endoscopic tool; and
a second connector configured to releasably connect said second component to an endoscope.

2. The surgical accessory of claim 1, wherein said variable length assembly comprises buckling resistance means for provide buckling resistance to the flexible portion of the flexible endoscopic tool that is received within at least one of said first component and said second component.

3. The surgical accessory of claim 1, wherein at least part of said first component is configured to slide within said second component.

4. The surgical accessory of claim 1, wherein at least one of said first component and said second component is substantially rigid.

5. The surgical accessory of claim 1, further comprising strain relief means for relieving strain between said variable length assembly and at least one of said first connector and said second connector.

6. The surgical accessory of claim 5, wherein said strain relief means is attached to said second component and said second connector is attached to said strain relief means.

7. The surgical accessory of claim 1, further comprising a spring, wherein said spring is attached to said second component and wherein said second connector is attached to said spring.

8. The surgical accessory of claim 1, further comprising a pivot member configured to allow said second component to articulate relative to said second connector.

9. The surgical accessory of claim 1, wherein said first connector is configured to releasably connect said first component to a control member of the flexible endoscopic tool.

10. The surgical accessory of claim 1, wherein said second connector is configured to releasably connect said second component to a proximal end of the endoscope.

11. The surgical accessory of claim 1, wherein said second connector is configured to releasably connect said second component to a working channel port of the endoscope.

12. The surgical accessory of claim 11, further comprising a seal member defining a passageway therein for insertably receiving the flexible portion of the flexible endoscopic tool, said seal member configured to at least resist gas passing therethrough when said second component is connected to the working channel port of the endoscope and when the flexible portion of the flexible endoscopic tool is insertably received in said passageway.

13. The surgical accessory of claim 11, further comprising sealing means for at least resisting gas passing therethrough when said second component is connected to the working channel port of the endoscope and when the flexible portion of the flexible endoscopic tool is insertably received in said sealing means.

14. A surgical accessory for guiding a flexible endoscopic tool, said surgical instrument comprising:
a variable length assembly comprising a first end and a second end, wherein said first end is movable relative to said second end, and wherein said variable length assembly is configured to receive a flexible portion of the flexible endoscopic tool;
a first connector configured to releasably connect said first end of said variable length assembly to the flexible endoscopic tool; and
a second connector configured to releasably connect said second end of said variable length assembly to an endoscope.

15. The surgical accessory of claim 14, wherein said variable length assembly further comprises a first component and a second component, wherein said first component is telescopically engaged with said second component, and wherein said first component and said second component are adapted to receive the flexible portion of the flexible endoscopic tool.

16. The surgical accessory of claim 15 wherein at least a portion of said first component is configured to slide within said second component.

17. The surgical accessory of claim 14, wherein said variable length assembly comprises buckling resistance means for provide buckling resistance to the flexible portion of the flexible endoscopic tool that is received by said variable length assembly.

18. The surgical accessory of claim 14, wherein said variable length assembly defines a longitudinal axis, wherein said first end is movable relative to said second end in a direction one of substantially parallel to and collinear with said longitudinal axis, and wherein said variable length assembly is substantially rigid in a direction substantially transverse to said longitudinal axis.

19. The surgical accessory of claim 14, wherein said variable length assembly is bendable without permanently deforming.

20. The surgical accessory of claim 19, wherein said variable length assembly comprises a coiled material.

21. The surgical accessory of claim 19, wherein said variable length assembly comprises a laser cut material.

22. The surgical accessory of claim 14, further comprising a strain relief mechanism, wherein said strain relief mechanism is attached to said second end of said variable length assembly and said second connector is attached to said strain relief mechanism.

23. The surgical accessory of claim 22, wherein said strain relief mechanism comprises a spring.

24. The surgical accessory of claim 14, wherein said first connector is configured to releasably connect said first end to a control member of the flexible endoscopic tool.

25. The surgical accessory of claim 14, wherein said second connector is configured to releasably connect said second end to a proximal end of the endoscope.

26. The surgical accessory of claim 14, wherein said second connector is configured to releasably connect said second end to a working channel port of the endoscope.

27. The surgical accessory of claim 26, further comprising a seal member defining a passageway therein for insertably receiving the flexible portion of the flexible endoscopic tool, said seal member configured to at least resist gas passing therethrough when said second end is connected to the working channel port of the endoscope and when the flexible portion of the flexible endoscopic tool is insertably received in said passageway.

28. The surgical accessory of claim 26, further comprising sealing means for at least resisting gas passing therethrough when said second component is connected to the working channel port of the endoscope and when the flexible portion of the flexible endoscopic tool is insertably received in said sealing means.

29. A surgical instrument, comprising:
a variable length assembly for guiding a surgical tool, said variable length assembly comprising a first end and a second end, wherein said first end is movable relative to said second end; and
a surgical tool for insertion into a patient's body, wherein said surgical tool is attachable to said first end of said variable length assembly, and wherein, when said surgical tool is attached to said first end, said variable length assembly is configured such that movement of said surgical tool causes movement of said first end of said variable length assembly relative to said second end.

30. A surgical instrument for insertion into a patient's body, said surgical instrument comprising:
a variable length assembly for guiding a surgical tool, said variable length assembly comprising a first end and a second end, wherein said first end is movable relative to said second end; and
an endoscope having an insertable portion and a non-insertable portion, wherein said first end of said variable length assembly is attached to said non-insertable portion of said endoscope.
